(19) United States
(12) Patent Application Publication
(10) Pub. No.: US 2014/0364857 A1
(43) Pub. Date: Dec. 11, 2014

(54) Joint Arthroplasty Devices, Systems, and Methods

(71) Applicant: ConforMIS, Inc., Bedford, MA (US)

(72) Inventors: Raymond A. Bojarski, Attleboro, MA (US); Thomas Minas, Dover, MA (US); Wolfgang Fitz, Sherborn, MA (US)

(21) Appl. No.: 14/373,569

(22) PCT Filed: Feb. 7, 2013
(86) PCT No.: PCT/US13/25216

§ 371 Date: Jul. 21, 2014

Related U.S. Application Data
(60) Provisional application No. 61/596,182, filed on Feb. 7, 2012.
(60) Provisional application No. 61/635,270, filed on Apr. 18, 2012.
(60) Provisional application No. 61/697,978, filed on Sep. 7, 2012.

(51) Int. Cl. A61B 17/16

(52) U.S. Cl. CPC A61B 17/1668; A61B 17/1662
USPC 606/89; 606/87

(57) ABSTRACT

Various devices, surgical tools, molds, methods and/or surgical techniques are disclosed herein that incorporate a variety of features to improve and/or simplify the preparation of a patient's anatomical surfaces for installation of joint implant replacement and/or resurfacing components.

### RELATED APPLICATIONS

This application claims the benefit of U.S. Provisional Patent Application No. 61/596,182 to Bojarski, entitled “Patient Selectable Joint Arthroplasty Devices and Surgical Tools,” filed Feb. 7, 2012. This application additionally claims the benefit of U.S. Provisional Patent Application No. 61/635,270 to Chao, entitled “Improved Tibial Guides, Tools, and Techniques for Resecting the Tibial Plateau,” filed Apr. 18, 2012. This application additionally claims the benefit of U.S. Provisional Patent Application No. 61/697,978 to Martin et al., entitled “Patient Selectable Joint Arthroplasty Devices and Surgical Tools,” filed Sep. 7, 2012. The entire contents of each of the three above-referenced U.S. provisional patent applications is incorporated herein by reference in its entirety.

### FIELD

The present disclosure relates to articular repair systems (e.g., resection cut strategy, guide tools, and implant components) as described in, for example, U.S. patent application Ser. No. 13/397,457, entitled “Patient-Adapted and Improved Orthopedic Implants, Designs And Related Tools,” filed Feb. 15, 2012, and published as U.S. Patent Publication No. 2012-0209394, which is incorporated herein by reference in its entirety. In particular, the present disclosure describes surgical tools, molds and/or surgical techniques incorporating a variety of features to improve and/or simplify the preparation of a patient's anatomical surfaces for installation of joint implant replacement and/or resurfacing components.

### BACKGROUND

The natural anatomical joint structures of an individual may undergo degenerative changes due to a variety of reasons, including injury, osteoarthritis, rheumatoid arthritis, or post-traumatic arthritis. When such damage or degenerative changes become far advanced and/or irreversible, it may ultimately become necessary to replace all or a portion of the native joint structures with prosthetic joint components. Joint replacement is a well-tolerated surgical procedure that can help relieve pain and restore function in injured and/or severely diseased joints, and a wide variety of prosthetic joints are well known in the art, with different types and shapes of joint replacement components commercially available to treat a wide variety of joint conditions.

As part of the surgical repair procedure for a total joint replacement, the underlying anatomical support structures are typically prepared to receive the joint implant components. For example, the placement of a femoral implant component can typically involve preparation of the caudad portion of the femoral bone (otherwise known as the distal head of the femur), which can include surgical resection of portions of the medial and femoral condyles of the femur, as well as the resection (e.g., cutting, drilling, rongeuring, scraping) of other anatomical features of the femur and/or surrounding soft tissues. This preparation will desirably create an anatomical support structure capable of accommodating and adequately supporting the femoral implant component or components, which is ultimately secured to the femur. Similar surgical steps can be performed to the tibia and/or the patella, as well as other anatomical structures, as necessary.

One or more surgical guide tools or jigs can be used to assist the surgeon in preparing the underlying anatomical support structure(s). There is a need, however, for improved surgical guide tools and jigs to increase the increase the efficiency and reproducibility of accurately preparing underlying anatomical support structure(s) for an implant.

### BRIEF DESCRIPTION OF THE SEVERAL VIEWS OF THE DRAWINGS

FIG. 1 depicts an embodiment of a first femur jig;

FIGS. 2 and 3 depict an embodiments of an alignment jig component including features for incorporation into a two-piece jig assembly;

FIG. 4 depicts one embodiment of a third guide tool or jig, which includes a removable anterior referencing feature or stylus;

FIG. 5 depicts an alternative embodiment of a third guide tool or jig which includes a removable and/or rotatable anterior referencing feature or wing;

FIGS. 6 and 7 depict an alternative embodiment of a third guide tool or jig configured to engage a removable flexion spacer;

FIGS. 8A through 8C depict another alternative embodiment of a third guide tool or jig which includes a removable flexion spacer.

### DETAILED DESCRIPTION

The following description is presented to enable any person skilled in the art to make and use the various embodiments of devices, concepts and methods described herein. Various modifications to the embodiments described will be readily apparent to those skilled in the art, and the generic principles defined herein can be applied to other embodiments and applications without departing from the spirit and scope of the present systems and methods as defined by the appended claims. Thus, the present disclosure is not intended to be limited to the embodiments shown, but is to be accorded the widest scope consistent with the principles and features disclosed herein. To the extent necessary to achieve a complete understanding of systems and methods disclosed, the specification and drawings of all issued patents, patent publications, and patent applications cited or referred to in this application are incorporated herein by reference.

3D guidance surgical tools, referred to herein as a 3D guidance surgical templates, that may be used for surgical assistance can include, without limitation, using templates, jigs and/or molds, including 3D guidance molds. It is to be understood that the terms “template,” “jig,” “mold,” “3D guidance mold,” and “3D guidance template,” shall be used interchangeably within the detailed description and any appended claims to describe the tool unless the context indicates otherwise.

A variety of traditional guide tools are available to assist surgeons in preparing a joint for an implant, for example, for resectioning one or more of a patient's biological structures during a joint implant procedure. However, these traditional guide tools typically are not designed to match the shape (contour) of a particular patient's biological structure(s). Moreover, these traditional guide tools typically are not designed to impart patient-optimized placement for the resection cuts. Thus, using and properly aligning traditional guide tools, as well as properly aligning a patient's limb (e.g., in rotational alignment, in varus or valgus alignment, or alignment in another dimension) in order to orient these traditional guide tools, can be an imprecise and complicated part of the implant procedure.

Certain embodiments described herein provide improved surgical guide tools and methods for preparing a patient's biological structure during a joint implant procedure. In various embodiments, 3D guidance surgical tools can include guide apertures. It is to be understood that the term guide aperture shall be used interchangeably within the detailed description and appended claims to describe guide surfaces, guide elements, limiter or shielding elements, captured cut guides, and/or uncaptured cut guides.

Various embodiments disclosed herein also include implants and procedures where the implant has an inner, bone-facing surface and an outer, joint-facing surface, and the inner, bone-facing surface engages an articular surface of a first biological structure (e.g., bone or cartilage) at a first interface. The articular surface can be a native surface, a cut surface, a preexisting implant component and/or various combinations and/or quantities/distributions thereof (e.g., multiple cut planes separated by a region of natural subchondral bone and/or articular cartilage). In addition, an outer, joint-facing surface on the component opposes a second, outer joint-facing surface on an opposing joint implant component at a joint interface. In certain embodiments, one or more features of the implant component, for example, various inner, bone-facing surfaces and/or various outer, joint-facing surfaces can be patient-adapted (i.e., comprising one or more patient-specific and/or patient-engineered features).

Various embodiments described herein include the use of a guide tool having at least one patient-specific bone-facing surface portion that substantially negatively-matches at least a portion of a biological surface at the patient's joint. The guide tool further can include at least one aperture for directing movement of a surgical instrument, for example, a securing pin or a cutting tool. One or more of the apertures can be designed to guide the surgical instrument to deliver a patient-optimized placement for, for example, a securing pin or resection cut. In addition or alternatively, one or more of the apertures can be designed to guide the surgical instrument to deliver a standard placement for, for example, a securing pin or resection cut. As used herein, “jig” also can refer to guide tools, for example, to guide tools that guide resectioning of a patient's biological structure. Alternatively, certain guide tools can be used for purposes other than guiding a drill or cutting tool. For example, balancing and trial guide tools can be used to assess knee alignment and/or fit of one or more implant components or inserts.

In various embodiments, the apertures, holes, guides and/or resection cut slots in a particular guide tool can be substantially, horizontal, substantially diagonal, or substantially vertical, for example, as compared to the patient's mechanical axis and/or anatomical axis. Moreover, one or more of the resection cut slots can allow for a complete resection cut or a partial resection cut, e.g., scoring of the patient's bone to establish a resection cut that can be finished after removing the tool. This approach can be advantageous by allowing for faster resection in the absence of the guide tool. Moreover, one or more resection cut slots can include a blade-depth or drill-depth stop. This is particularly useful for step resection cuts, for example, vertical step resection cuts, that connect two facets or planes of a resected surface.

Various embodiments disclosed herein include systems, methods, and devices for performing a series of bone cuts to receive a patient-adapted implant. Specifically, a set of jigs can be designed in connection with the design of a patient-adapted implant component. The designed jigs can guide the surgeon in performing one or more patient-adapted cuts to the bone so that those cut bone surface(s) negatively-match patient-adapted bone-facing surfaces of corresponding patient-adapted implant components. Some of the embodiments of the jigs described herein can be used for a femur-first surgical cut technique.

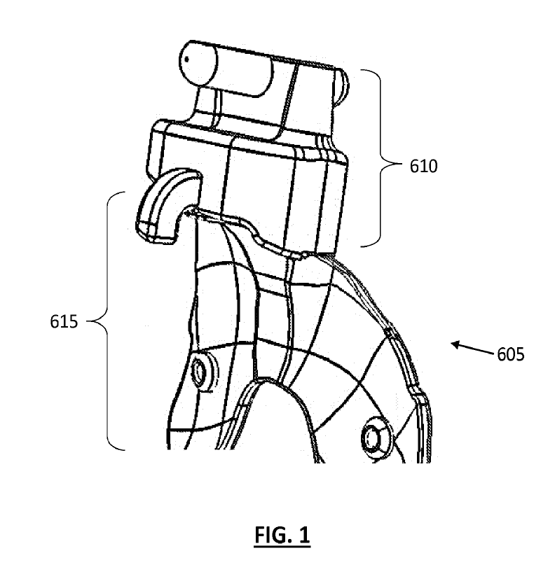

In some embodiments, a first step can include the use of a first femoral jig to establish peg holes and/or pin placements for subsequent jigs (e.g., a jig used for a distal cut). FIG. 1 shows an illustrative embodiment of a first jig 615. The first jig may be designed to circumvent at least a portion of cartilage. For example, the first jig may be specifically designed to circumvent cartilage of a particular thickness (e.g., 3 mm), and in some embodiments, the particular thickness may be based on patient-specific information. The first jig may incorporate an inner surface that substantially conforms to some, or all, of the outer surface of the uncut distal femur (e.g., cartilage and/or bone), whereby the jig fits onto the femur in a predetermined position and orientation. In various embodiments, the jig can comprise a flexible material which allows the jig to flex and “snap fit” around the distal femur. In addition, the inner surface of the jig may be designed to avoid and/or accommodate the presence of osteophytes and other anatomical structures on the femur. One or more guide-pin openings extending through the surface of the jig can provide position and orientation guidance for guide pins that can be inserted into the distal surface of the femur. After insertion of the guide pins, the first jig may then be removed from the femur.

In some embodiments, after insertion of one or more guide pins and removal of the first jig, a cannulated drill and/or coring tool can be used over one or more of the pins. The drill and/or coring tool can be used to remove all, or at least a portion, of cartilage adjacent to the pins, which can expose the subchondral bone surface adjacent to the pins. Because subchondral bone can be readily visualized through various imaging methods, and because subchondral bone is significantly rigid, it can provide a reliable reference surface for the placement of additional jigs.

Various alternative embodiments of first jigs can be utilized. For example, the jig may include one or more features for referencing or registering any osteophyte or other bone or joint deformity at the intended surgical site. The jig may further include the following illustrative features, which may facilitate fewer surgical steps: 1) small bosses or protrusions that touch off within the cored out cartilage area referencing to sub-condylar bone and 2) two anterior pin apertures that can be placed to house and/or reference a distal cutting jig (not shown). In various embodiments, the jig may further include a small boss or other features that can be used to contact and/or visually reference subcondylar bone or other anatomical structures on an anterior portion of the femur.

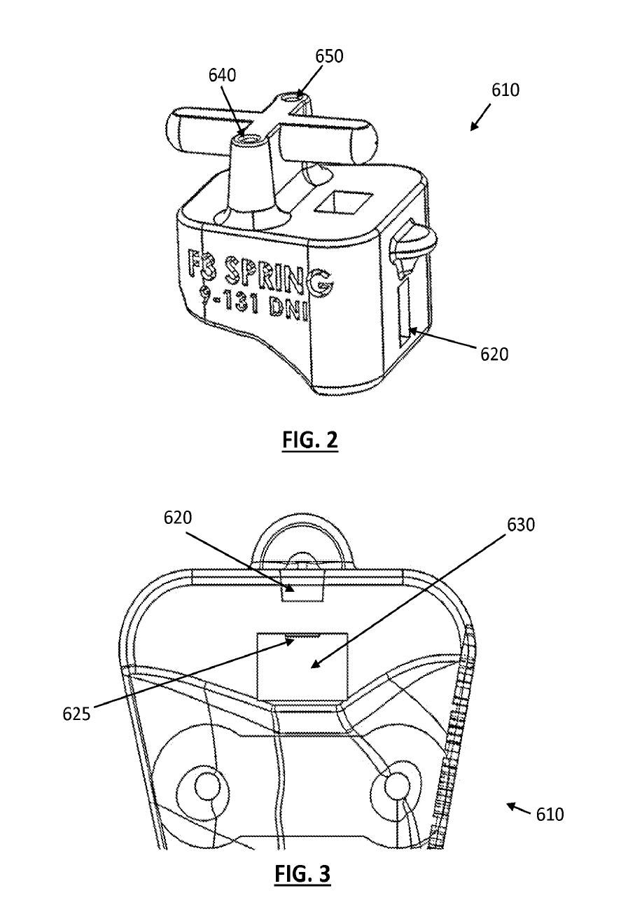

FIGS. 2 and 3 depict various views of an optional, exemplary alignment jig component 600, which includes features that can be incorporated into a two-piece jig assembly 605 with first jig 615. The alignment jig component 600 can be an anterior section 610 of the two-piece assembly 605, and can be separated from a posterior portion 615 that includes various patient-specific features, as desired. The component 600 can include a securement mechanism or spring feature 620 which desirably secures the component 600 to a corresponding post or shaft (not shown) on the posterior portion 615. A portion 625 of the spring feature 620 can protrude into an opening 630 designed for fitment onto and/or over an attachment mechanism on another component or jig, which allows connection and alignment of the alignment jig with the other component or jig. When the attachment mechanism is fitted through the opening, it can engage the protruding side of the spring feature, resulting in a compression of the spring feature and tighter fit between the components/jigs. If desired, this removable feature can be included on one or more jigs, which can facilitate alignment of such jigs to the two anterior pin apertures 640 and 650 and associated anchoring pins (not shown) previously secured to the femur.

In various embodiments, after positioning of the first jig and/or alignment jig, as discussed above, a distal femoral cut jig may be positioned. In some embodiments, the distal femoral cut jig can be configured to engage the first jig and/or alignment jig in a predetermined position and orientation. In some embodiments, the distal femoral jig may include holes for receiving pins positioned and placed via the first jig and/or alignment jig (e.g., pins placed via holes 640 and 650) in a predetermined position and orientation. In some embodiments, after positioning the distal femoral cut jig, other jigs may be removed from the femur, and one or more (e.g., to create a stepped cut) cuts may be made on the distal femur, guided by one or more guiding surfaces of the distal femoral cut jig. Once the distal femoral cut(s) has been made, the distal femoral cut jig may be removed.

In various embodiments, once a distal femoral cut has been made, the tibia may be cut using one or more jigs designed to make cuts on the proximal tibia. In some embodiments, such tibial cuts may be patient-adapted. One or more tibial cuts may be used to prepare a cut tibial plateau. In certain embodiments, tibial jigs can be designed to accommodate for composite thickness from the distal cut femur. Alternatively or additionally, a balancing chip can be used to address differences in the distance between the tibia and femur surfaces. For example, in certain embodiments a tibial jig may be designed to rest on 2 mm of cartilage, while a balancing chip is designed to rest on the distal cut femur.

In some embodiments, after a cut tibial plateau surface has been prepared, one or more balancer chips and/or spacer blocks may be utilized to assess the joint for appropriate balance. For example, in some embodiments an extension spacer may be inserted between the cut distal femur and the cut proximal tibia, and with the knee in extension, varus and valgus stress may be applied by the surgeon to assess the joint balance. Additionally or alternatively, the knee may be brought into flexion, a flexion spacer may be placed on the cut tibial plateau, and varus and valgus stress may be applied by the surgeon to assess the joint balance. The flexion spacer may be configured such that when positioned on the cut tibial plateau, un-cut posterior portions of the femoral condyles may rest on the superior surface(s) of the spacer. As discussed further below, shims of various sizes may be attached to the surface of the spacer(s) to account for fit issues (e.g., if there is posterior cartilage loss) to increase the composite size of the spacer. In some embodiments, proper joint balance may be indicated by observing the joint space opening approximately 1-2 mm medially and laterally with the application of stress. If the knee is balanced in flexion but tight in extension, this may indicate that additional bone (e.g., 2 mm) should be removed from the distal femur. If the knee is tight in flexion and extension, this may indicate that additional bone (e.g., 2 mm) should be removed from the proximal tibia. If the knee is balanced in extension and tight in flexion, this may indicate that osteophytes are impinging on the PCL, and slope may need to be added to the proximal tibia.

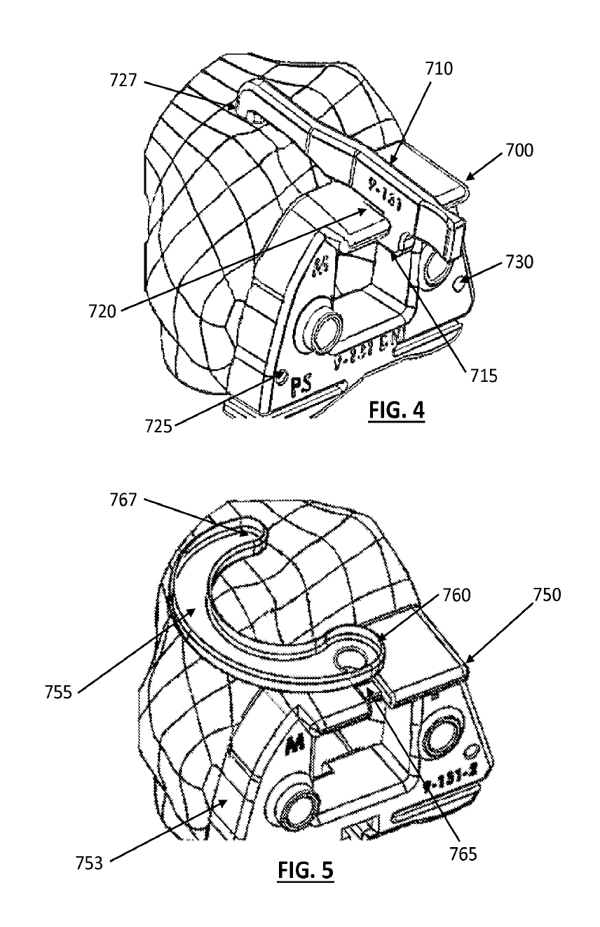

In some embodiments, after the tibial plateau has been cut (and optionally, after insertion of one or more spacers) a third guide tool or third jig may be used to cut anterior and/or posterior portions of the femur. Optionally, a flexion spacer may be/remain positioned on the cut tibial plateau during use of the third jig. FIG. 4 depicts an exemplary embodiment of a third guide tool or third jig 700. In some embodiments, the third jig 700 may include a moveable and/or removable anterior referencing feature or stylus 710. The stylus 710 may include a hook feature 715 which engages with a matching notch feature 720 of the jig 700. The stylus 710 further includes a patient-specific distal end 727, which desirably contacts or otherwise references an anatomical feature of the femur when the jig 700 is in a desired position. The anterior-referencing stylus can be designed and/or selected using patient-specific anatomical information, which can drive various features of the stylus, including the shape and position of the surface at the end of the stylus that contacts the bone surface (which in many cases will be uncut subchondral bone and/or have minimal cartilage thereon) as well as the length and/or angulation of the stylus. The stylus desirably helps positioning the A-P cut guide, which in turn can determine the resection depths of the various anterior, posterior and chamfer cuts. The use of this embodiment of a stylus can be implemented based on the femoral implant design, and can include various selectable and/or built-in and/or surgeon desired rotation values. Once the jig has been properly positioned, a pair of anchoring pins or other devices can be secured through openings 725 and 730 in the jig 700, the stylus 710 can be removed from the third jig 700, and the various guiding surfaces and/or other features of the jig 700 can be utilized to prepare and resect the patient anatomy.

FIG. 5 depicts an alternative embodiment of a third guide tool or third jig 750, which includes a removable and rotatable anterior referencing feature or wing 760. In this embodiment, the wing 760 includes a curved body 755 having an enlarged stem (not shown) which engages with a matching notch feature 765 of the jig 750. The wing 760 further includes a patient-specific distal end 767, which desirably contacts or otherwise references an anatomical feature of the femur when the jig 750 is in a desired position. In a manner similar to the previously described embodiment, the anterior-referencing wing can be designed and/or selected using patient-specific anatomical information, which can drive various features of the wing, including the shape and position of the surface at the end of the wing that contacts the bone surface (which in many cases will be uncut subchondral bone and/or have little articular cartilage or soft tissues thereon) as well as the length, curvature and/or angulation of the wing. In alternative embodiments, the wing 760 may be non-patient specific, and used simply to verify alignment of the anterior portion of the jig 750 to a desired location on an anterior portion of the femoral shaft. The wing desirably helps positioning the third jig, which in turn can determine the resection depths of the various anterior, posterior and chamfer cuts. In some embodiments, the wing 760 may be used by the surgeon to confirm that an anterior resection cut (e.g., guided by third jig 750) will not notch the femur. In many embodiments, the shape of the curved body 755 can facilitate use of the tool to avoid various osteophytes and/or other anatomical features between the jig 750 and the desired location on an anterior or other portion of the femoral shaft.

In some embodiments, the peripheral sides (e.g., medial side 753 of third jig 750) of various jigs can be sized and shaped to align with the outer margins of the cut bone surface, providing an additional alignment and reference guide to assure the surgeon the procedure is proceeding as planned. In some embodiments, the various cut guide jigs described herein can be “linked” or referenced to each. The jigs may be linked in such a manner by, for example, incorporating extra pin apertures and/or drill holes on the various jigs to position one or more extra pins, optionally in a posterior location, which aligns with an appropriate pin aperture on a subsequent cut guide, thereby allowing cross-referencing of positions and/or alignments between various tools.

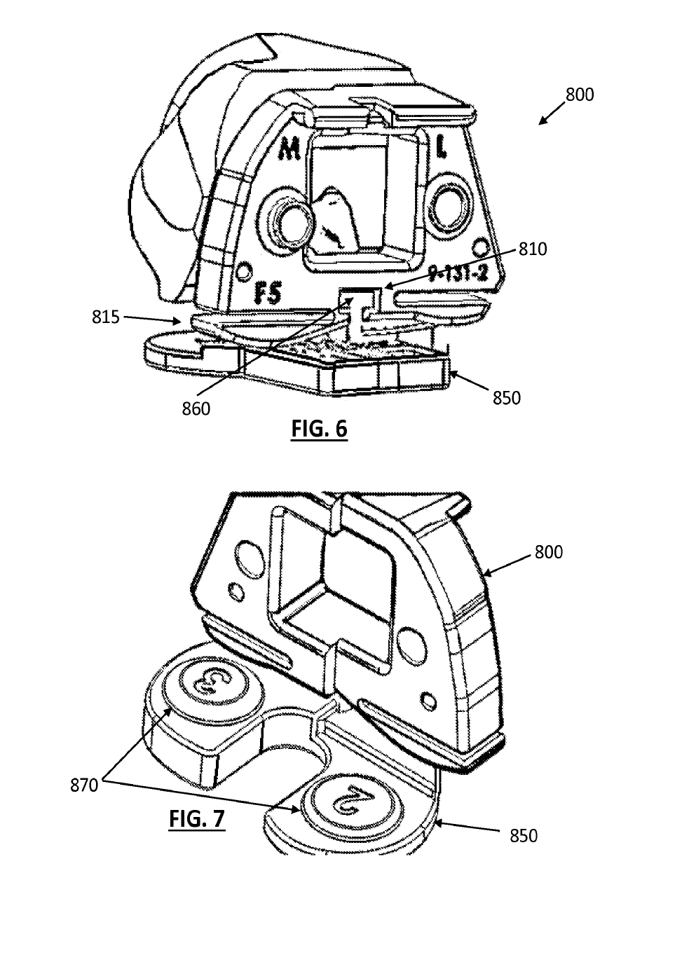

FIGS. 6 and 7 depict one alternative embodiment of a third guide tool or third jig 800. The third jig 800 may be configured for releasably engaging a flexion spacer 850. A connection mechanism such as a slot 810 can be formed in a posterior portion 815 of the jig 800, which can be configured to secure a corresponding protrusion or “T” 860 on the flexion spacer 850. In various embodiments, the flexion spacer may include a variety of removal washers or shims 870 of various thicknesses, shapes, angulations and/or sizes.

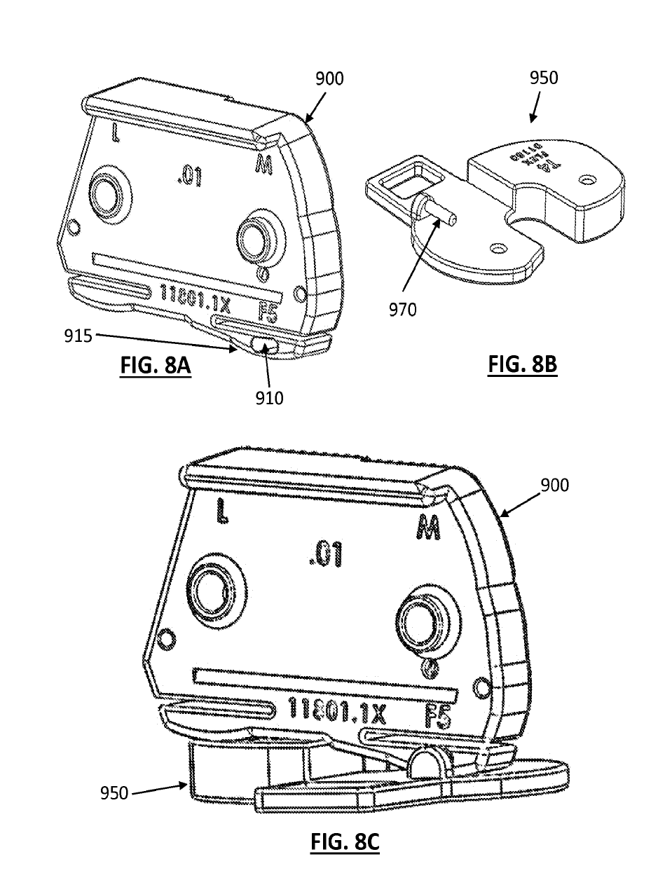

FIGS. 8A through 8C depict another alternative embodiment of a third guide tool or third jig 900. The third jig 900 can be configured to releasably engage a flexion spacer 950. For example, the third jig 900 can include a connection mechanism, such as, for example, a slot 910, formed adjacent a posterior portion 915 of the jig 900, and the flexion spacer 950 may include a corresponding connection mechanism, such as, for example, protrusion 970 extending from a surface of the flexion spacer 950. Depending upon the width and/or orientation of slot 910, some rotation and/or lateral movement of the spacer 950 relative to the jig 900 when the jig 900 is secured to the distal head of the femur. As previously noted, a flexion spacer 850, 950 may include and/or be configured to receive a variety of removable washers or shims of various thicknesses, shapes, angulations and/or sizes. In some embodiments, the connection mechanism can be positioned on the medial side (as shown in FIGS. 8A-C) which can allow for slight rotation of the third jig 900 with respect to the medial side. In various embodiments, if a varus/valgus deformity of the knee is observed, realignment can be addressed by including added thickness to the balancing chip in the area that would produce a leg in neutral alignment. For a grossly malaligned contra-lateral leg, correction can be per a surgeon's order. If desired, the balancing chip could include a feature to attach it to a tibial jig and thereby allow for accurate distal placement of a tibial cut while at the same time accommodating for composite thickness.

The various descriptions contained herein are merely exemplary in nature and, thus, variations that do not depart from the gist of the teachings are intended to be within the scope of the teachings. Such variations are not to be regarded as a departure from the spirit and scope of the teachings, and the mixing and matching of various features, elements and/or functions between various embodiments is expressly contemplated herein. One of ordinary skill in the art would appreciate from this disclosure that features, elements and/or functions of one embodiment may be incorporated into another embodiment as appropriate, unless described otherwise above. Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

What is claimed is:

1. A jig for use in a surgical procedure to repair a joint of a patient, the jig comprising:
a guide aperture configured to accommodate a surgical tool for cutting or drilling into a portion of tissue of the joint; and
a connection mechanism configured to releasably engage a guide tool at a predetermined position and orientation,
wherein the guide aperture has a position and orientation in relation to the connection mechanism that defines a predetermined cutting or drilling path into the portion of tissue when the jig and connection mechanism are connected via the connection mechanism and the jig is positioned on a first joint surface and the guide tool is positioned on a second joint surface.

2. A system for use in a surgical procedure to repair a joint of a patient, the system comprising:
the jig of claim 1;
a guide tool having a connection mechanism configured to releasably engage a corresponding connection mechanism of the jig at a predetermined position and orientation; and
one or more shims of a predetermined size and configured for releasably attaching to a surface of the guide tool.

3. The jig of claim 1 or system of claim 2, wherein the portion of tissue of the joint comprises one or more of femoral tissue, tibial tissue, subchondral bone, and cartilage.

4. The jig of claim 1 or system of claim 2, wherein the portion of tissue comprises a posterior portion of a femoral condyle.

5. The jig of claim 1 or system of claim 2, wherein the first joint surface comprises a distal femoral cut surface and the second joint surface comprises a proximal tibial cut surface.

6. The jig of claim 1 or system of claim 2, wherein the connection mechanism is positioned on a medial side of the jig and is configured to permit rotation of the jig relative to the guide tool.

7. The jig of claim 1 or system of claim 2, wherein the jig includes a side surface that is sized and shaped to substantially align with at least a portion of the outer margin of the first joint surface.

8. A method of making the jig of claim 1 or the system of claim 2.
